(19) United States
(12) Patent Application Publication
(10) Pub. No.: US 2003/0035827 A1
(43) Pub. Date: Feb. 20, 2003

(54) Pharmaceutical compositions containing substrates of enabling enzymes to preferentially deliver drugs away from PBVC or GIC systems

(76) Inventor: David Lew Simon, Mansfield Center, CT (US)

Correspondence Address: David Lew Simon, P.O. Box 618, Mansfield Center, CT, 06250, US

(21) Appl. No.: 09/928,854

(22) Filed: Aug. 13, 2001

(51) Int. Cl. A61K 9/70

(52) U.S. Cl. 424/449

(57) ABSTRACT

Compositions are described for linking drugs or pharmaceutical agents to substrates such that the drug or pharmaceutical agent can be rendered relatively ineffective when not exposed to an enabling enzyme contained within a compartment of a living animal or human.

### BACKGROUND

1. Field of Invention

This invention relates to pharmaceutical preparations for the delivery of drugs and/or medications, specifically for providing drug delivery compositions that preferentially deliver drugs or medications other than via the enteral or alimentary route and allowing for preferential delivery of the drug or medication by routes of administration directed more directly to blood plasma containing compartments.

2. Description

Pharmaceutical drug delivery systems are essential components to safe and effective delivery of medications to humans and animals. Various such systems have been developed to allow for delayed drug delivery, drug delivery sustained over a period of time, delivery of an ideal dose of a drug and so forth. Various routes of administration are described, as for example via mouth (a.k.a. per os) or rectum thus entering the digestive system or alimentary canal, and parenteral routes that do no come into direct contact with the alimentary tract such as intravenous administration, transdermal administration, subcutaneous administration, intramuscular administration, transmucosal administration, etc. Most, if not all, commercially available medicinal preparations are formulated for either enteral (usually per os or “p.o.”) administration or parenteral administration. There are no known commercially available pharmaceutical compositions prepared with the idea that a drug can be administered by either the enteral or parenteral route(s) with the intention that delivery by one mechanism (e.g. parenteral) will be favored over the other mechanism (e.g. enteral).

Recently, it has been generally reported that a drug delivery system has been invented that incorporates a “bead” containing a drug that is imbedded in a matrix whereby the matrix dissolves over time releasing its contents into the alimentary tract while the drug-containing bead passes relatively unaltered through the alimentary tract in a way in which said drug is not released in significant amounts. However, if the drug delivery system is physically disrupted, as by crushing, the drug is released from the bead, allowing for deposition of a free form of the drug into any compartment into which the drug delivery system is administered. The drug delivery system that is derived from this concept is herein and hereafter referred to as the “physical crushing drug delivery system” (or “PCDDS”).

The present invention provides significant advantages over the PCDDS because the present invention does not depend on physical disturbance such as crushing to make it easier to ingest. In fact, the making of pills or tablets easier to ingest by any means, including crushing, is a common and important method for administration of p.o. medications. Some patients are inherently unable to swallow whole tablets. Such patients include small children, adults with swallowing disorders, individuals who have undergone a previous cerebrovascular accident—especially that which involves the basal ganglia or brainstem, surgical patients whose jaws are wired shut and who must ingest medication through a drinking straw, as well as patients who require delivery of food or medications through a feeding tube such as through a nasogastric tube or a surgically placed tube into the stomach, duodenum or small intestine. In addition, there are individuals without physical disability that are so impaired by emotional or psychological distress that they are unable to swallow whole hard pills. Further, without regard to patient ability to ingest a drug p.o., there are some circumstances where administration of a whole hard tablet is less desirable than administering a more malleable form of a medicinal preparation. One such example is a patient predicted to be non-compliant with medication regimens. Such patients are notorious for creating an illusion of ingestion of a whole tablet when they are observed being administered the medication, only to hide the pill under the tongue, or between the cheek and teeth and so forth, to spit out the tablet at a later time when the patient is no longer observed. Examples of such patients may include a psychologically disturbed patient that through illness does not realize the necessity to have the medication administered, or a substance abusing patient that wishes to continue to abuse a pleasure-producing drug and is therefore motivated to not ingest medication treatments aimed at reducing or eliminating the abuse of the pleasure-producing drug.

Common examples of medications in tablet form that are typically crushed prior to administration include the crushing and subsequent ingestion of aspirin for prophylaxis against an impending heart attack (myocardial infarction), and the crushing of baby aspirin so it may be mixed in liquid or soft food prior to administration to a child. In fact, these types of medication administration are commonplace.

### OBJECTS & ADVANTAGES OF THE PRESENT INVENTION

Accordingly, besides the objects and advantages of the compositions for preferential parenteral administration of pharmaceuticals described in my patent above, the following objects and advantages of the present invention are:

(a) To allow for distinction between a drug better delivered parenterally than enterally based upon physiological properties of the body compartment to which it is delivered, rather than based on the hardness or physical properties of a drug-containing bead;

(b) To allow for a medicinal drug to be more biologically active if administered parenterally than if administered through the alimentary tract;

(c) To Limit the biological activity of an agonist drug if is administered in composition with an antagonizing drug such that if the two drug composition is administered enterally the antagonizing drug will have little effect relative to the agonist drug, but when administered parenterally the antagonizing drug will have pharmacologically therapeutic effect relative to the agonist drug;

(d) The object and advantage of (c) above that is independent from crushing the two drug composition;

(e) To allow for a medication formulation or pharmaceutical preparation containing a scheduled narcotic drug that is rendered less likely to be used illicitly by parenteral administration.

Further objects and advantages will become apparent from a consideration of the ensuing description and drawings.

### DRAWINGS & FIGURES

FIG. 1 shows the basic chemical notation to describe the breaking of an ester bond with the addition of water, known as hydrolysis.

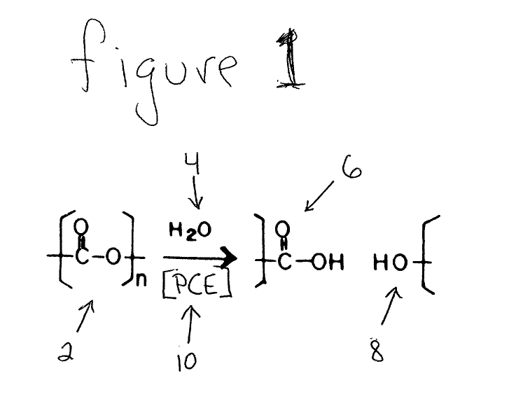

### REFERENCE NUMERALS IN DRAWING & FIGURES

2 notation representative of an intact polyester with intact ester bond

4 water molecule

6 organic acid group of one side of a hydrolyzed ester moiety within a “broken” polyester

8 organic base of the other side of a hydrolyzed ester moiety with a “broken” polyester

10 pseudocholinesterase or plasma cholinesterase or butyrocholinesterase (all equivalent terms) in solution with water and the polyester

### DESCRIPTION

The present invention is based upon the selective availability of naturally occurring physiological enzymes, generally known as esterases, in certain body compartments of the living organism. The prototype enzyme is pseudocholinesterase, plasma cholinesterase or butyrocholinesterase, (referred to hereafter as “PCE”). This enzyme is found prominently in the blood plasma compartment with red blood cells. Despite being manufactured in the liver, generally considered part of the digestive system, it is not characteristically bioavailable within the human or animal alimentary canal, and if found in the alimentary canal or gastrointestinal compartment (hereafter the “GI” compartment), it differs in concentration from the plasma compartment significantly enough to have physiologically distinguishing activities in the plasma compartment as compared to the GI compartment. When present in the GI canal PCE is present almost exclusively in longitudinal muscle, and not appreciably in the epithelial cells or intestinal villi. These differences were less pronounced in the stomach where G-cells rich in PCE are found (J Clin Chem Clin Biochem. Vol. 20, 1982, pp. 69-74), however, the compartmental milieu of the stomach does not allow for appreciable physiologic activity of PCE there (see next paragraph). More plainly stated, PCE is typically found in biologically relevant concentration in the plasma compartment, whereas the bioequivalence of PCE is significantly different in the GI compartment. This difference in bioequivalence of PCE in the plasma compartment as compared to the GI compartment can be exploited within the context of the present invention.

Further, PCE requires an optimal milieu in which to function. Specifically, it is only an effective catalyst within an optimal pH range. In the stomach compartment, the milieu is much too acidic for significant biological activity of PCE. In other parts of the GI compartment the amount of time PCE is in direct contact with its substrate is not sufficient enough for PCE to fully exert its effects. In the blood plasma compartment, however, the physiological buffering system in such that the milieu presents an optimal pH for PCE enzymatic activity, in addition to PCE being in direct contact with its substrate for sufficient periods as the substrate circulates throughout the blood. Even when corrected for pH, the enzymatic activity of PCE in plasma is at least several-fold greater in the blood plasma compartment than in the GI compartment (Gen. Pharmac. Vol. 14, No. 4, pp. 459-60, 1983; and J Invest Dermatol September 1979; 73(3):239-42).

Therefore, there are at least three distinct reasons relating to concentration, pH and the time PCE is in direct contact with its substrate, as to why PCE is physiologically effective in the blood plasma compartment but not the GI compartment. Nevertheless, these preferential actions of PCE depending upon its location within the human body have not been appreciated by those skilled in the art, rendering the present invention non-obvious.

A typical embodiment of a composition for preferential parenteral administration of pharmaceuticals is now described. A matrix containing a benzodiazepine such as diazepam is composed. Within the matrix is a linked benzodiazepine antagonist such flumazenil. The flumazenil is linked to a PCE-susceptible ester in any of a number of ways The linkage may be in the form or one or more ester bonds, chemically linking the flumazenil to an ester or ester derivative with pharmaceutical properties such that the linked flumazenil is not easily absorbed across the endothelial lining of the GI compartment. Such pharmaceutical properties may include lipid solubility, net ionic charge, molecular weight, or any number of parameters known to influence transit of molecules across gastrointestinal epithelium or endothelial lining of the GI compartment.

Alternatively, the flumazenil may be coated with PCE-susceptible esters by any number of methods readily available to one skilled in the art. This may be accomplished with polyester microcapsules consisting of a linear ester bond susceptible to PCE. Any of a variety of known biodegradable polymers may be used providing that an acceptable threshold of PCE-susceptible ester bonds is present. Such polyester matrices have been made of D,L-Lactic acid, L-Lactic acid, glycolic acid and so forth. Polyesters have long been used for bioabsorbable sutures as commonly used during human surgery. It was generally appreciated by those skilled in the art that these polyesters degrade by polymer hydration due to altering hydrogen bonding and van der Waals forces (Controlled Release of Drugs, ed. Morton Rosoff 1989, ISBN 0-89573-321-8, p. 79). The novelty of the present invention is supported by that fact that despite the long use of dissolvable sutures since 1970, the role of esterases in polyester hydrolysis was not appreciated by those skilled in the art.

Matrix biodegration of polyesters generally involves bulk erosion. Thus by incorporating flumazenil within a PCE-susceptible polyester matrix, as the PCE catalyzes ester hydrolysis the flumazenil will be released as the polyester erodes. If the polyester is designed such that PCE-susceptible ester linkages are optimally abundant, a polyester matrix can be designed and manufactured such that the flumazenil will be released to a much greater extent when in the presence of PCE, as in the blood plasma compartment, than when biologically active PCE is not present in sufficient amounts to cause catalyzation of physiologically significant ester hydrolysis, as in the GI compartment. There are now various readily commercially available computer software programs that can aid in the design of such a polymer matrix with optimally abundant PCE-susceptible linkages that in light of the present invention, the design and manufacture of such a matrix should be readily apparent to one skilled in the art.

Substrates for which PCE is enzymatically active and to which flumazenil may be linked include acetylthiocholine, butyrylthiocholine, propionylthiocholine and succinylmonocholine as examples. The ideal substrate for linkage is one that does not cause significant adverse pharmacological events when administered in the amounts suggested by the present invention. Ideally, the substrate should be enzymatically degraded in plasma at a very fast rate, especially in comparison to rate of enzymatic reaction in the GI compartment (if any). The table below lists known substrates of PCE with each substrate's Kₘ and k_{cat} as determined by previous experiments:

| | | |
|---|---|---|
| SUBSTRATE | Kₘ | K_{cat} |

| | | | |
|---|---|---|---|
| 1-NAPHYLACETATE | 370 | uM | 36,000/min. |
| Acetylcholine | 9,000 | uM | 33,200/min. |
| Aspirin | 1,600 | uM | 7,200/min. |
| Benzoylcholine | 22 | uM | 15,000/min. |
| Butyrylcholine | 1,700 | uM | 80,000/min. |
| Butyrylthiocholine | 3.8 & 0.4 | mM | 30,000/min. |
| Hepanoylcholine | 1,110 | uM | 35,000/min. |
| Heroin | 450 | uM | 500/min. |
| Hexanoylcholine | 820 | uM | 36,000/min. |
| Methylprednisolone acetate | — | | 25/min. |
| O-Nitrophenylbutyrate | 400 | uM | 48,000/min. |
| Pentanoylcholine | 1,500 | uM | 63,000/min. |
| Procaine | 100 | uM | 255/min. |
| Propionylcholine | 3,100 | uM | 55,000/min. |
| Succinyldithiocholine | 1,080 | uM | 600/min. |
| Tetracaine | 8 | uM | 74/min. |

### Sources: Pharmacology & Therapeutics 1990 (47):35-60 & Molecular Pharmacology 1996 (50):1423-31

In another embodiment of the invention, a drug with opioid antagonist properties, such as naloxone, naltrexone, nalmefene, buprenorphine, cholecystokinin, pentazocine, butorphanol, nalbuphine, 6-alpha naltrexol, 6-beta-naltrexol, naloxol, 6-beta-naltrexamine, naltrindole, TIPP peptides (e.g. TIPP H-Tyr-Tic-Phe-OH, TIPP-psi {H-Tyr-Tic-[CH₂NH]-Phe, Phe-OH}), SoRI9409 or analogues of 17-substituted-6,7-dehydro-4,5-alpha-epoxy-3,14-dihydroxy-6,7:2′,3′-indolomorphinans {e.g. the N-alkyl analogues (N-ethyl through N-heptyl), branched N-alkyl chain analogues (N-isopropyl, N-2-methylpropyl, and N-3 methylbutyl), and N-alkenyl analogues ((E)-N-3-methylallyl (crotyl), N-2-methylallyl, and N-3,3-dimethylallyl}, referred to hereafter as simply the “antagonist,” can be contained within a matrix susceptible to PCE hydrolysis but resistant to typical digestive enzymes. The antagonist-containing PCE susceptible matrix can be suspended in a matrix that is not susceptible to PCE but which is susceptible to typical digestive enzymes that also contains an opioid agonist analgesic such as oxycodone. When the totally compounded medication consisting of antagonist in the PCE-susceptible matrix suspended in the other non-PCE susceptible matrix containing oxycodone (hereafter the “Pharmaceutical Composition”) is administered enterally, the PCE-susceptible matrix, in absence of PCE, is left relatively unaltered such that the antagonist is not released for absorption through the intestinal mucosa, while simultaneously the oxycodone is released for absorption through the intestinal mucosa. However, if the Pharmaceutical Composition were to be administered parenterally, as for example during intravenous injection as commonly practiced by opioid substance abusers, then the PCE-susceptible matrix will undergo hydrolysis, thus releasing the antagonist into the plasma blood volume compartment. The agonist would therefore tend to negate the oxycodone, such that the oxycodone, when administered parenterally, is rendered relatively ineffective as compared to when it is administered parenterally. The Pharmaceutical Composition can be in any of a number of forms, including a gel contained within a gelatin capsule, a liquid, a paste or a variety of other forms commonly administered by p.o. route. Because the present invention can be applied in any one of these malleable forms, the Pharmaceutical Composition may be administered through a feeding tube or to those individuals that cannot swallow whole solids. This is because the present invention does not depend so much on hardness of the material surrounding the antagonist as it depends more on whether the material linked to the antagonist is susceptible to the catalyst action on ester hydrolysis by PCE. This is a very important distinction between the present invention and the PCDDS prior art. In the PCDDS compound, the whole hard pill form only acts to release one drug (e.g. oxycodone) and not the other drug (e.g. the antagonist) IF it is not crushed. If it is crushed, however, then both drugs will be released, even if administered p.o. The present invention, in a gel capsule embodiment, will release only the one drug (e.g. oxycodone) and not the second drug (e.g. the antagonist). This allows easy ingestion of the present invention while delivering the antagonist away from gastrointestinal absorption, which cannot be accomplished with the PCDDS technology that crushes the bead along with the rest of the pill.

In a third embodiment of the invention, a pharmaceutical composition is made such that an orally administered form of a medication which has a smaller therapeutic window when administered parenterally compared to enteral administration, can be formulated to reduce the likelihood of life-threatening adverse effects when prescribed for self-administration by psychologically unstable humans that tend to self-mutilate or otherwise harm themselves. An example of this is an anticholinergic agent, such as atropine or glycopyrrolate, contained within a PCE-susceptible and GI resistant matrix, that is further suspended in another matrix that is PCE-resistant and GI susceptible which contains a beta-blocker that undergoes significant first past metabolism in the liver. When administered orally, the beta-blocker's bioavailability is significantly less than when administered parenterally, bypassing the liver and “first pass” effect. If such a beta-blocker needed to be prescribed for a cardiac condition to a self-destructive psychiatric patient on an outpatient basis, as commonly occurs, it is possible if not probable that the patient would attempt to self-administer the beta-blocker intravenously in an attempt to commit suicide. Higher than therapeutic dose of beta-blockers can cause cardiac arrest. Within the context of the present invention described in this third embodiment above, the anticholinergic would be released which would tend to negated the dangerous effects of the beta-blocker. In general, beta-blockers suppress the heart rate, or cause slowing of the heart or total cardiac standstill. Anticholinergics such as atropine and glycopyrrolate cause an increase in heart rate. Thus, when administered intravenously the anticholinergic prophylactically treats what would otherwise be an overdose of beta-blocker. Other examples can be formulated to include psychiatric medications and their antidotes within the scope of the present invention.

The example embodiments of the present invention noted above are for illustrative purposes only, and are not intended to limit the scope of the invention. For instance, the invention is not limited to polyesters that are susceptible to PCE. The invention also is not limited to esters susceptible to PCE. In fact, other enzymes occurring with biological activity in the plasma blood volume compartment of greater magnitude as compared to the biological activity within the GI compartment may be exploited within the scope of the present invention. For the purpose of illustration, the following definitions are given:

i.) “PBVC-predominant” enzyme is defined as a naturally occurring enzyme within an animal or human that has biological activity under naturally occurring conditions (taking into account body temperature, pH of the milieu within the compartment, etc.) much greater in the plasma blood volume compartment than is the biological activity in the GI compartment. An example, for illustrative purposes only, is butyrocholinesterase.

ii.) “GIC-predominant” enzyme is defined as a naturally occurring enzyme within an animal or human that has biological activity under naturally occurring conditions (taking into account body temperature, pH of the milieu within the compartment, etc.) much greater in the alimentary or gastrointestinal tract lumen compartment than is the biological activity in the plasma blood volume compartment. Examples, for illustrative purposes only, include papain, pepsin, trypsin and chymotrypsin.

iii.) “PBVC-predominant” substrate is defined as a naturally occurring enzyme substrate within an animal or human that has biological susceptibility to an enzyme under naturally occurring conditions (taking into account body temperature, pH of the milieu within the compartment, etc.) much greater in the plasma blood volume compartment than is the biological susceptibility in the GI compartment. Examples, for illustrative purposes only, include succinylcholine and the local anesthetic known as chloroprocaine.

iv.) “GIC-predominant” substrate is defined as a naturally occurring enzyme substrate within an animal or human that has biological susceptibility to an enzyme under naturally occurring conditions (taking into account body temperature, pH of the milieu within the compartment, etc.) much greater in the alimentary or gastrointestinal tract lumen compartment than is the biological susceptibility in the plasma blood volume. Examples, for illustrative purposes only, include various peptides that are routinely degraded by digestive enzymes.

v.) “PBVC-resistant” substrate is defined as a substrate within an animal or human that has biological resistance to a naturally occurring enzyme under naturally occurring conditions (taking into account body temperature, pH of the milieu within the compartment, etc.) within the plasma blood volume compartment.

vi.) “GIC-resistant” substrate is defined as a substrate within an animal or human that has biological resistance to a naturally occurring enzyme under naturally occurring conditions (taking into account body temperature, pH of the milieu within the compartment, etc.) within the gastrointestinal compartment.

vii.) “Enabling Enzyme” is defined as an enzyme that catalyzes the degradation of a substrate such that the substrate's degradation allows release of an active chemical, drug or pharmaceutical agent into a biologically significant compartment.

A fourth embodiment of the invention allows preferential delivery of a medicinal drug into plasma blood volume compartment relative to the GI compartment. The utility of this embodiment is seen within the context of pediatric medications. There are some medications intended to be delivered parenterally to children for a variety of reasons. There may be alternative methods of drug delivery that are easier for children to tolerate, especially those children that cannot easily swallow or those that choose not to because of the behavioral characteristics of childhood. Such an alternative methods may include inhalation of an aerosol, administration of a nasal spray, ophthalmologic preparation (“eye drops”), or sublingual administration. The alternative method may provide a means of bypassing the liver such that “first pass” metabolism of the drug does not occur. This may be important, not just in terms of pharmacokinetics and drug delivery of the medicinal drug itself, but certain drugs requiring administration to children may be particularly toxic to the liver, therefore it is very much preferred to decrease delivery of the drug to the liver to the greatest extent possible. This can be accomplished by avoiding alimentary or p.o. delivery of the medicinal drug by means of such alternative methods of parenteral drug delivery. However, small children are notorious for getting into places that could be potentially harmful, such as medicine chests. A child could very well stray into a cabinet or medicine chest where the parenteral medicinal drug is kept. The child could orally self-administer or ingest the liver-toxic parenterally intended medication. This could have serious, life-threatening adverse effects on the child. A way to “child-proof” the pharmaceutical preparation containing the liver-toxic medicinal drug would be to link the drug to a polymer that is not easily degraded in the GI compartment, but which is easily enough degradable in the plasma blood volume compartment. This can be accomplished within the scope of the present invention. The liver toxic drug could be coated with a PBVC-predominant substrate. This may be accomplished by any one of a variety of methods currently known to one skilled in the art, including but not limited to, various microencapsulation techniques. That is, the liver toxic drug may be encapsulated with the PBVC-predominant substrate, and the encapsulated drug may then be incorporated into a medium optimized for parenteral delivery depending upon the intended delivery method (e.g. aerosol, hydrogel, etc.).

Another embodiment of the invention allows preferential delivery of a medicinal drug into GI compartment relative to the plasma blood volume compartment. There may be some drugs with a sufficiently small therapeutic window that when administered parenterally could result in a toxic or fatal dose of the drug. However, because of first pass metabolism or decreased bioavailability with the drug when administered enterally or by the p.o. or per rectum routes, toxicity due to the drug's administration is greatly reduced. Therefore, it would be very important to formulated a pharmaceutical composition where the therapeutic drug can be absorbed readily through the alimentary canal or GI compartment, but which is not as easily absorbed available in the blood plasma volume compartment. The utility of this embodiment is seen within the context of pediatric medications. Small children are notorious for getting into places that could be potentially harmful, such as medicine chests. A child could very well stray into a cabinet or medicine chest where the therapeutic drug is kept. The child could accidentally spray an aerosol into his or her eye, allowing for significant parenteral absorption (as an ophthalmologic pharmaceutical might be absorbed, for example). Alternatively, the aerosol could be accidentally sprayed about the child's face in such a way that the child could inhale a significant amount of the therapeutic drug with a narrow therapeutic window, thus resulting in a large bolus dose that could potentially be very toxic. This could have serious, life-threatening adverse effects on the child. A way to “child-proof” the pharmaceutical preparation containing the therapeutic drug would be to link the drug to a polymer that is not easily degraded in the plasma blood volume compartment, but which is easily enough degradable in GI compartment.

### SUMMARY

Accordingly, the reader will see that the compositions linking drugs or pharmaceutical agents to substrates of enabling enzymes can result in:

targeting of the drug or pharmaceutical agent to a specific physio-anatomical compartment within a human or animal;

relative exclusion of drug or pharmaceutical agent from a specific physio-anatomical compartment within a human or animal;

Decreased overall toxicity of drugs or pharmaceutical agents in animals and humans;

Increased therapeutic window for medicinal drugs and pharmaceutical medications;

Improved safety of medications for children;

Decreased abuse of narcotic drugs such as benzodiazepines and opioid agonist analgesics.

I claim by letters patent:

1.) A pharmaceutical composition for preferentially delivering drugs away from the gastrointestinal compartment of an animal or human comprising
a drug,
a PBVC-predominant substrate,
and a GIC-resistant substrate.

2.) A pharmaceutical composition for preferentially delivering drugs away from the plasma blood volume compartment of an animal or human comprising
a drug,
a GIC-predominant substrate,
and a PBVC-resistant substrate.

3.) The pharmaceutical composition in claim 1 where the PBVC-predominant substrate and the GIC-resistant substrate are one and the same substrate.

4.) The pharmaceutical composition in claim 2 where the GIC-predominant substrate and the PBVC-resistant substrate are one and the same substrate.

5.) The pharmaceutical composition in claim 3 where the drug is linked to the substrate.

6.) The pharmaceutical composition in claim 4 where the drug is linked to the substrate.

7.) The pharmaceutical composition in claim 5 where the linkage of the drug to the substrate is by way of organic bond.

8.) The pharmaceutical composition in claim 6 where the linkage of the drug to the substrate is by way of organic bond.

9.) The pharmaceutical composition in claim 5 where the linkage of the drug to the substrate is by way of microencapsulation of the drug with the substrate.

10.) The pharmaceutical composition in claim 6 where the linkage of the drug to the substrate is by way of microencapsulation of the drug with the substrate.

11.) The pharmaceutical composition of claim 1 where the drug is one from the class of benzodiazepine antagonists or opioid antagonists.

12.) The pharmaceutical composition of claim 1 where the drug is one from the class of benzodiazepine antagonists or opioid antagonists.

13.) The pharmaceutical composition of claim 1 where the PBVC-predominant substrate's enabling enzyme is an esterase.

14.) The pharmaceutical composition of claim 13 where the enabling enzyme is PCE.

15.) The pharmaceutical composition of claim 3 where the drug is one of any of the drugs naloxone, naltrexone, nalmefene, buprenorphine, cholecystokinin, pentazocine, butorphanol, nalbuphine, 6-alpha naltrexol, 6-beta-naltrexol, naloxol, 6-beta-naltrexamine, naltrindole, TIPP peptides (e.g. TIPP H-Tyr-Tic-Phe-OH, TIPP-psi {H-Tyr-Tic-[CH₂NH]-Phe, Phe-OH}), SoRI9409 or analogues of 17-substituted-6,7-dehydro-4,5-alpha-epoxy-3,14-dihydroxy-6,7:2′,3′-indolomorphinans {e.g. the N-alkyl analogues (N-ethyl through N-heptyl), branched N-alkyl chain analogues (N-isopropyl, N-2-methylpropyl, and N-3 methylbutyl), and N-alkenyl analogues ((E)-N-3-methylallyl (crotyl), N-2-methylallyl, and N-3,3-dimethylallyl}.

16.) The pharmaceutical composition of claim 9 where the drug is one of any of the drugs naloxone, naltrexone, nalmefene, buprenorphine, cholecystokinin, pentazocine, butorphanol, nalbuphine, 6-alpha naltrexol, 6-beta-naltrexol, naloxol, 6-beta-naltrexamine, naltrindole, TIPP peptides (e.g. TIPP H-Tyr-Tic-Phe-OH, TIPP-psi {H-Tyr-Tic-[CH₂NH]-Phe, Phe-OH}), SoRI9409 or analogues of 17-substituted-6,7-dehydro-4,5-alpha-epoxy-3,14-dihydroxy-6,7:2′,3′-indolomorphinans {e.g. the N-alkyl analogues (N-ethyl through N-heptyl), branched N-alkyl chain analogues (N-isopropyl, N-2-methylpropyl, and N-3 methylbutyl), and N-alkenyl analogues ((E)-N-3-methylallyl (crotyl), N-2-methylallyl, and N-3,3-dimethylallyl}.

17.) The pharmaceutical composition of claim 16 where the enabling enzyme is an esterase.

18.) The pharmaceutical composition of claim 17 where the esterase is PCE.

19.) The pharmaceutical composition of claim 9 where the drug is flumazenil.

20.) A pharmaceutical composition for preferentially delivering drugs away from the gastrointestinal compartment of an animal or human comprising
a drug (“Drug A”),
a PBVC-predominant substrate and a GIC-resistant substrate which are one and the same
a matrix in which the drug linked to the substrate is suspended
another drug (“Drug B”) within said matrix where Drug A and Drug B counteract one another.

21.) The pharmaceutical composition in claim 20 where
Drug A is one of any of the drugs naloxone, naltrexone, nalmefene, buprenorphine, cholecystokinin, pentazocine, butorphanol, nalbuphine, 6-alpha naltrexol, 6-beta-naltrexol, naloxol, 6-beta-naltrexamine, naltrindole, TIPP peptides (e.g. TIPP H-Tyr-Tic-Phe-OH, TIPP-psi {H-Tyr-Tic-[CH₂NH]-Phe, Phe-OH}), SoRI9409 or analogues of 17-substituted-6,7-dehydro-4,5-alpha-epoxy-3,14-dihydroxy-6,7:2′,3′-indolomorphinans {e.g. the N-alkyl analogues (N-ethyl through N-heptyl), branched N-alkyl chain analogues (N-isopropyl, N-2-methylpropyl, and N-3 methylbutyl), and N-alkenyl analogues ((E)-N-3-methylallyl (crotyl), N-2-methylallyl, and N-3,3-dimethylallyl}
Drug B is an opioid agonist analgesic

22.) The pharmaceutical composition of claim 21 where the opioid agonist analgesic is oxycodone.

23.) The pharmaceutical composition of claim 21 where the enabling enzyme is an esterase.

24.) The pharmaceutical composition of claim 23 where the esterase is PCE.
